(19) United States
(12) Patent Application Publication
(10) Pub. No.: US 2025/0225296 A1
(43) Pub. Date: Jul. 10, 2025

(54) HIGH-PRECISION PREDICTION METHOD FOR SHELF-LIFE OF AQUATIC PRODUCTS UNDER VARIABLE TEMPERATURE ENVIRONMENT

(71) Applicants: QINGDAO UNIVERSITY OF SCIENCE AND TECHNOLOGY, Qingdao (CN); Qingdao Kodak Future Biotechnology Co.Ltd, Qingdao (CN)

(72) Inventors: Wancui XIE, Qingdao (CN); Kuan LU, Qingdao (CN); Xihong YANG, Qingdao (CN); Sheng GAO, Qingdao (CN); Lin SONG, Qingdao (CN)

(73) Assignees: QINGDAO UNIVERSITY OF SCIENCE AND TECHNOLOGY, Qingdao (CN); Qingdao Kodak Future Biotechnology Co.Ltd, Qingdao (CN)

(21) Appl. No.: 18/807,730

(22) Filed: Aug. 16, 2024

(30) Foreign Application Priority Data
Jan. 10, 2024 (CN) 202410032045.0

(51) Int. Cl. G06F 30/27; G01N 33/12

(52) U.S. Cl. CPC G06F 30/27; G01N 33/12; G16C 20/30

(57) ABSTRACT

A method for high-precision prediction of shelf-life of aquatic products under a variable temperature environment includes: constructing and training a deep learning-based Facformer model for shelf-life prediction; obtaining aquatic products to be predicted; determining the temperature, TPC (Total Plate Count), and the TVB-N(Total Volatile Base-Nitrogen) of the aquatic products in a consecutive p-day period; recording the temperature data of the aquatic products in a period of days p to q, and importing the temperature in a period of consecutive p days, the TPC, and the TVB-N, and the temperature data within p to q days were imported into a pre-trained Facformer model for inference to obtain the predicted values of TPC, TVB-N, and shelf-life of aquatic products under variable temperature environment.

### CROSS-REFERENCE TO RELATED APPLICATIONS

This application claims priority to Chinese Patent Application No. 202410032045.0, filed on Jan. 10, 2024, the content of which is incorporated herein by reference in its entirety.

### TECHNICAL FIELD

The present application relates to the technical field of food safety, and in particular relates to a method for high-precision prediction of shelf-life of aquatic products under variable temperature environment.

### BACKGROUND

Aquatic products are not only a source of high-quality proteins, unsaturated fatty acids, etc., but are also rich in functional factors beneficial to the human body, such as active peptides, EPA (Eicosapentaenoic Acid), DHA (Docosahexaenoic Acid), polysaccharides and trace elements, which occupy an important position in the structure of the human diet. However, aquatic products are highly susceptible to the influence of microorganisms, endogenous enzymes and changes in the external environment during transport, processing and storage, which reduces the freshness of the products and leads to spoilage, resulting in huge wastage, and the fuzzy prediction of the true shelf-life of aquatic products is one of the key reasons for their wastage.

Traditional shelf-life prediction models for aquatic products are mainly based on the determination of characteristic indexes such as TVB-N (Total Volatile Base-Nitrogen) and TPC (Total Plate Count), and then the characteristic indexes are directly fitted statistically to determine shelf-life under different constant temperature environments by adopting the zero-order or first-order kinetic equations such as the Arrhenius model, etc. However, at this stage, the acquisition of the basic data required by the traditional models has a long time period, and the prediction is mostly based on historical experience and the results are easily affected by storage temperature. However, at this stage, the long time period required for obtaining the basic data of traditional models, the reliance on the fitted values based on historical experience and the susceptibility of the results to the influence of storage temperature, etc., lead to the results of the traditional shelf-life prediction is only a vague reference value, which is especially obvious when solving the problem of long-range shelf-life prediction.

In addition, although the prediction of shelf-life of aquatic products under different temperatures can be achieved based on traditional models such as Arrhenius, the predicted value is based on the case of constant storage temperature in the future, whereas the storage temperature of aquatic products is often a constantly changing value due to the influence of various factors during the actual storage process, so if the aquatic products are under good storage temperatures, their actual circulatable period may be far beyond the predicted shelf-life, but usually destroyed early because the predicted period has been reached. Therefore, if the fish is stored at a good temperature, its actual circulation period may lag far behind the predicted shelf-life, but it will usually be destroyed earlier because it has reached the predicted period. On the contrary, if aquatic products are stored at undesirable temperatures, they will deteriorate prematurely, bringing serious quality and safety risks, which is the main reason why no shelf-life model has been able to achieve accurate prediction of aquatic products' shelf-life so far.

### SUMMARY

In order to solve the problems existing in the prior art, the present application provides a high precision prediction method for shelf life of aquatic products in a variable temperature environment, comprising:
pre-constructing and training a Facformer model for predicting shelf life of aquatic products;
obtaining an aquatic product to be predicted;
measuring the temperature, TPC and TVB-N of the aquatic product during a total of p consecutive days and recording them;
recording the temperature data of the aquatic product from p to q (q>p) days, and importing the data of the temperature, TPC and TVB-N in a total of p consecutive days, and the temperature data from p to q days into the pre-trained Facformer model for inference, in order to obtain predicted values of the TPC and TVB-N of the aquatic product under variable temperature conditions;
inputting limiting standards for TVB-N and TPC in the aquatic product into the Facformer model, and comparing with the predicted TPC and TVB-N of the aquatic product to obtain a predicted value of the shelf-life of the aquatic product under variable temperature conditions.

Optionally, the pre-constructing and training the Facformer model works in the following steps:
firstly, preparing the aquatic product by removing the shell, viscera and bone spurs of the aquatic product to obtain edible part;
next, placing the prepared aquatic product in constant temperature incubators at m random temperatures in a range from 4° C. to 40° C., sampling and determining the TPC and TVB-N of the samples under variable temperature conditions at a fixed time every day for n consecutive days, to obtain a time series of data comprising m×n×2 entries;
then, dividing the time series data into a training dataset, a validation dataset, and a test dataset, and standardizing them to establish a time series dataset;
finally, defining the Facformer model in a Python™ 3.7 language environment, wherein the Facformer model mainly consists of three parts: a data Embedding, an Encoder and a Decoder.

The data Embedding layer of the Facformer model works in the following steps:
the numerical embedding module is used to convert the measured values of TPC and TVB-N of aquatic products in the time-series data set into a matrix language that can be recognized by the model, and the positional embedding module is used to arrange the measured values of TPC and TVB-N of aquatic products according to the sequential order of the storage time in the matrix, which is converted into a matrix language with the characteristics of time series (step 10); if the measured data are discrete variables, the weight information of the measured values is transformed into the matrix language that can be recognized by the model through Relu loss function, feed-forward neural network and cyclic coding layer (step 11); if the measured data are continuous variables, the weight information of the measured values is directly transformed into the matrix language that can be recognized by the model through the feed-forward neural network and the embedding layer with linear transformation (step 12);
in summary, through the data embedding layer processing, the measured values with location information are transformed into Xᵢ feature matrix by the model (step 11), the values of days, temperature and other indicators are transformed into XMᵢ feature matrix by the model (step 12), and the values containing the measured values, days, temperature and other indicators are transformed into XXMᵢ feature matrix by the model (step 13), and finally, after the output of the encoder, the measured values, days, temperature and other indicator values are transformed into XPᵢ, XPMᵢ and XXPMᵢ feature matrices by the model.

The steps for building the Encoder for the Facformer model are as follows:
firstly, the encoder of the Facformer model consists of three parallel encoder modules a, b and c, which are the inputs of the Facformer model; after the original time-series dataset is obtained from the feature matrices through the data embedding layer, the respective feature matrices are linearised into matrices Q (step 21), matrix K (step 22) and matrix V (step 23) through different linear transformation matrices W_{Q}, W_{K} and W_{V}, respectively, which are used to record their own feature information, and subsequently the model will perform dot product operation on matrix Q and matrix K to obtain the weight matrix containing the weights of the total number of aquatic product colonies TPC and TVB-N for scoring, and the weighting coefficients matrix is obtained by normalising and downscaling the weights matrix, multiplied by the matrix V corresponding to each position, and summed to obtain the output matrix encoded by the single-head self-attention mechanism (step 24);
finally, in order to increase the computing speed of the Facformer model, the output matrix Zᵢ obtained from a total of h parallel single-head self-attention mechanisms was summed up, i.e., a comprehensive encoded matrix was obtained after h parallel operations of the self-attention mechanisms, and the comprehensive encoded matrix was firstly uplifted by the fully-connected layer in the feed-forward neural network and downlifted by invoking the Relu function, and the downlifted matrix was then standardized based on the normalisation layer (step 25); after that, the normalised matrix is normalised based on the normalisation layer, and the robustness of the model is increased by adding the residual neural network and the original feature input matrix, and finally a matrix-encoded query dictionary of aquatic products time-series dataset is obtained, which contains the detailed weight information of aquatic products' storage time, storage temperature, TPC and TVB-N during the storage period (step 26);
where Xᵢ feature matrix is transformed by encoder-a to get Kₐ and Vₐ matrices (step 27); XMᵢ feature matrix is transformed by encoder-b to get K_{b} and V_{b} matrices (step 28);
the XXMᵢ feature matrix is encoder-c converted to obtain the K_{c} and V_{c} matrices for subsequent decoding in the decoder (step 29).

The steps for building the Decoder for the Facformer model are as follows:
firstly, the Facformer's decoder consists of three parallel decoder modules, each containing at least two self-attention mechanisms for parsing the weight information of the matrix, and is the predictor and output side of the Facformer model;
next, the output matrices XPᵢ and XXPMᵢ obtained by the encoder are transformed into matrices Q, K and V through two different linear transformation layers, in which the matrix Q and K with the weight information of the samples will be masked by the masking matrix to remove the ambient noise that may affect the prediction results of the samples, and after dimensionality reduction, the masked prediction matrix is obtained (step 31);
then, the Qₐ matrix and Q_{c} matrix generated when the masking processed prediction matrix passes through the masking self-attention mechanism layer are passed downward, and the actual weight information of the data to be predicted is calculated by the second layer of the self-attention mechanism with the matrix K and matrix V passed in the encoder, and the actual weight information is compared with the query lexicon obtained from the encoding layer to obtain the prediction matrix; the Qₐ matrix generated by XPᵢ will be combined with the Kₐ matrix and Vₐ matrix passed by encoder-a for decoding of the message (step 32), and the Q_{c} matrix generated by XXPMᵢ will be combined with the K_{c} matrix and V_{c} matrix passed by encoder-c for decoding of the message (step 33);
after that, the output matrix XPMi obtained from the embedding layer for the number of days, temperature and other metrics is not masked with a masking matrix, and after it passes through the linear transformation layer, it passes directly through the self-attention mechanism layer to pass the Q_{b} matrix, which carries only the information about the future temperature without the rest of the ambient noise information, to the lower layer, and the Q_{b} matrix will be predicted by the three parallel arithmetic mechanisms to the quality metrics of the aquatic products in the future under the real variable-temperature conditions: firstly, the Q_{b} matrix will be combined using the matrix Kₐ passed in encoder-a and the matrix V_{b} passed in encoder-b through the Fac-self-attention mechanism, and at the same time, the matrix K_{b} passed in encoder-b and the matrix Vₐ passed in encoder-a will be used, and the combination of the two will be used to jointly compute the actual weight information of the data to be predicted, and the weight information will be used to compare with the query obtained from the encoding layer dictionary to obtain the prediction matrix; secondly, XPMᵢ uses a masking matrix to remove all the ambient noise through masking when inputting the first layer of multi-head self-attention mechanism, and the Q_{b} matrix generated when passing through the masking self-attention mechanism layer will be used to calculate the actual weight information of the data to be predicted through the second layer of the self-attention mechanism together with the matrix K_{b} and matrix V_{b} passed through the encoder, which will be used as a corrective matrix to increase the accuracy of the prediction; finally, the Q_{b} matrix is used for direct data fitting to the data to be predicted through convolutional neural networks and is used as a correction matrix to improve the accuracy of the model predictions (step 34);
finally, the output matrices XPᵢ, XPMi and XXPMᵢ obtained by the decoder will respectively elevate the dimensions of the matrices through the feed-forward neural network so as to make a comprehensive prediction of the data in a more detailed way, and the prediction weight information contained in the prediction matrices will be re-mapped to the time-series values through the normalisation layer, the linear transformation layer and the dimensionality reduction layer to obtain the prediction results, so as to complete the prediction of aquatic product TPC, TVB-N and shelf-life of aquatic products (step35).

The steps to determine the Facformer model are as follows:
firstly, the training dataset is fed into the Facformer model during the training phase to train the model hyperparameters;
next, the validation dataset is fed into the trained Facformer model during the validation phase to fine-tune the hyperparameters;
finally, the Facformer model was obtained by inputting the test dataset into the constructed Facformer model during the testing phase and evaluating the model prediction performance using MAE (Mean Absolute Error), MSE (Mean Squared Error), RMSE (Root Mean Squared Error), MAPE (Mean Absolute Percentage Error) and MSPE (Mean Squared Percentage Error).

Optionally, the determination of TVB-N in an aquatic product is carried out in the following steps:
firstly, a quantity of the aquatic product is taken and placed in a distillation tube, where distilled water is added and fully shaken and then macerated for a period of time;
next, using the automatic Kjeldahl method, magnesium chloride was added to the distillation tube, which was immediately connected to the distiller, and the parameters of the instrument were set as follows: boric acid to accept the volume of the liquid, distillation time or distillation volume, 0.1000 mol/L hydrochloric acid was used as the standard titration solution, and the end point was judged by using self-calibrating potentiometric titration, and the instrument was cleaned in time after the determination was completed;
the TVB-N in aquatic products was calculated according to the following formula:

$$TVB - N ( mg / 100 g ) = \frac{( {V}_{1} - {V}_{2} ) \times c \times 14}{m} \times 100 ,$$

where V₁ is the volume of hydrochloric acid standard titration solution consumed by the test solution, V₂ is the volume of hydrochloric acid standard titration solution consumed by the reagent blank, c is the concentration of hydrochloric acid standard titration solution, 14 is the mass of nitrogen equivalent to the titration of 1 mL of hydrochloric acid [c (HCl)=1.000 mol/L] standard titration solution, M is the mass of the sample, and 100 is the conversion factor.

Optionally, the TPC for determining the TPC in an aquatic product is carried out according to the following steps:
firstly, taking a certain amount of aquatic products into a sterile homogenising bag containing saline, homogenising for a period of time to make a 1:10 sample homogenate, and preparing a diluted sample homogenate in accordance with the 10-fold serial dilution method;
next, selecting 2˜3 dilutions of sample homogenate, aspirating the sample homogenate in a sterile petri dish, pouring the plate with counting agar medium at 46° C. and setting a blank control, and placing the plate at 30° C. after the agar solidifies;
finally, recording the number of plate dilutions and the corresponding number of colonies, and selecting the plates with TPC between 30 CFU (Colony-Forming Units) and 300 CFU and with continuous dilution for counting, the TPC in aquatic products was calculated according to the following formula:

$$TPC ( C F U ) = \frac{Σ C}{( {n}_{1} + 0.1 {n}_{2} ) d}$$

where is the sum of the number of plate colonies, n1 is the number of low dilution plates, n2 is the number of high dilution plates, and d is the low dilution dilution factor.

With the adoption of the above technical solution, the present application has at least the following beneficial effects:
firstly, the built-in multi-head self-attention mechanism of the Facformer model makes it parallel and fast, with less historical data used for inference, less time span required for data collection, shorter cycle time, and high experimental reproducibility;
secondly, the Facformer model performs shelf-life prediction through uninterrupted autonomous learning, which significantly improves the accuracy compared to traditional data-fitting based shelf-life prediction models;
lastly, the Facformer model based on the marking information constraint mechanism and the optional masking mechanism, which is not affected by the ambient temperature and can transform the temperature change of aquatic products in the future storage and transport process into the feature matrix and act synergistically with the historical data, is able to realise the accurate prediction of the shelf life of aquatic products in the variable-temperature environment.

### BRIEF DESCRIPTION OF THE DRAWINGS

In order to more clearly illustrate the technical solutions in the embodiments or prior art of the present application, the accompanying drawings to be used in the description of the embodiments or prior art will be briefly introduced below, and it will be obvious that the accompanying drawings in the following description are only some of the embodiments of the present application, and that for the person of ordinary skill in the field, other attachments can be obtained on the basis of the accompanying drawings, without paying creative labour.

FIG. 1 shows the overall framework diagram of the Facformer model.

FIG. 2 shows a network structure diagram of the data embedding layer of the Facformer model.

FIG. 3 shows a network structure diagram of the encoder of the Facformer model.

FIG. 4 shows a network structure diagram of a decoder of the Facformer model.

FIG. 5 shows the TPC prediction results of shrimp paste at several different temperature conditions during the testing phase of the Facformer model, where (a) to (k) are the TPC prediction results of shrimp paste at 4° C., 8° C., 12° C., 16° C., 20° C., 22° C., 24° C., 28° C., 32° C., 36° C., and 40° C. during the testing phase of the Facformer model, respectively.

FIG. 6 shows the TVB-N prediction results of shrimp paste at several different temperature conditions during the testing phase of the Facformer model, where (a) to (k) are the TPC prediction results of shrimp paste at 4° C., 8° C., 12° C., 16° C., 20° C., 22° C., 24° C., 28° C., 32° C., 36° C., and 40° C. during the testing phase of the Facformer model, respectively.

FIG. 7 shows the TPC prediction results of shrimp paste under several different temperature conditions based on the TPC prediction model by first-order kinetic equation, where (a) to (k) are plots of TPC prediction results for shrimp paste at 4° C., 8° C., 12° C., 16° C., 20° C., 22° C., 24° C., 28° C., 32° C., 36° C., and 40° C. for TPC prediction models constructed based on first-order kinetic equations, respectively.

FIG. 8 shows the TVB-N prediction results of shrimp paste under several different temperature conditions based on the TPC prediction model by first-order kinetic equation, where (a) to (k) are plots of TVB-N prediction results for shrimp paste at 4° C., 8° C., 12° C., 16° C., 20° C., 22° C., 24° C., 28° C., 32° C., 36° C., and 40° C. for TVB-N prediction models constructed based on first-order kinetic equations, respectively.

FIG. 9 shows the predicted results of quality indexes of shrimp paste at 24° C. in the actual prediction stage of the Facformer model, (A) shows the predicted results of TPC of shrimp paste at 24° C. in the actual prediction stage of the Facformer model, and (B) shows the predicted results of volatile salt base nitrogen TVB-N of shrimp paste at 24° C. in the actual prediction stage of Facformer model.

FIG. 10 shows the quality index prediction results of shrimp paste at 24° C. in the actual prediction stage of the Facformer model compared with the true value, (A) shows the TPC prediction results of shrimp paste at 24° C. in the actual prediction stage of the Facformer model compared with the true value, and (B) shows the TVB-N prediction results are plotted against the true values.

FIG. 11 shows the TPC prediction results of the TPC of grass carp at several different temperature conditions during the testing phase of the Facformer model, where (a) to (d) are the TPC prediction results of the TPC of grass carp at 4° C., 20° C., 36° C., and 40° C., respectively, during the testing phase of the Facformer model.

FIG. 12 shows the predicted results of TVB-N for grass carp at several different temperature conditions during the testing phase of the Facformer model, where (a) to (d) are the predicted results of TVB-N for grass carp at 4° C., 20° C., 36° C., and 40° C., respectively, during the testing phase of the Facformer model.

FIG. 13 shows the plots of TPC prediction results of TPC of grass carp at several different temperature conditions based on the TPC prediction model of TPC constructed based on first-order kinetic equations, where (a) to (d) are the plots of the TPC prediction results of TPC of grass carp at 4° C., 20° C., 36° C., and 40° C. based on the TPC prediction model of TPC constructed based on the first-order kinetic equation, respectively.

FIG. 14 shows the TVB-N prediction results of grass carp at several different temperatures based on the TVB-N prediction model constructed by the first-order kinetic equation, where (a) to (d) are the TVB-N prediction results of grass carp at 4° C., 20° C., 36° C. and 40° C., respectively, constructed by the TVB-N prediction model based on the first-order kinetic equation.

FIG. 15 shows the predicted results of quality indicators of grass carp at 20° C. during the actual prediction stage of the Facformer model, (A) shows the predicted results of TPC of grass carp at 20° C. during the actual prediction stage of the Facformer model, and (B) shows the predicted results of TVB-N of grass carp at 20° C. during the actual prediction stage of the Facformer model.

FIG. 16 shows the predicted results of quality indicators of grass carp at 20° C. in the actual prediction stage of the Facformer model compared with the true values, (A) shows the predicted results of TPC of grass carp at 20° C. in the actual prediction stage of the Facformer model compared with the true values, (B) shows the predicted results of VSN (Volatile Saline Nitrogen) in the actual prediction stage of the Facformer model compared with the true values of VSN (in the actual prediction stage of grass carp at 20° C. TVB-N prediction results are plotted against the true values.

### DETAILED DESCRIPTION OF THE EMBODIMENTS

The technical solutions in the embodiments of the present application will be described clearly and completely in the following in conjunction with the accompanying drawings in the embodiments of the present application, and it is obvious that the described embodiments are only a part of the embodiments of the present application and not all of the embodiments. Based on the embodiments in the present application, all other embodiments obtained by a person of ordinary skill in the art without making creative labour fall within the scope of protection of the present application.

In recent years, machine/deep learning algorithms based on time series prediction such as Random Forests, Recurrent Neural Networks, Convolutional Neural Networks, and Autoformer have been parsing subtle features of samples from multidimensional data through uninterrupted autonomic learning to significantly reduce potential fitting errors during dynamical analyses, and to robustly perform a variety of tasks, such as global weather prediction, earthquake epicenter detection, and rainfall prediction etc.

However, the current research of machine learning or deep learning algorithms in the field of aquatic products shelf-life prediction is almost blank, lacks of effective theoretical support and systematic research, and has not yet found any kind of model that can achieve accurate prediction of aquatic products shelf-life in variable temperature environments.

Therefore, the inventors envisage the application of machine/deep learning to the field of shelf-life prediction of aquatic products, and believe that there is an urgent need at this stage to establish a deep learning algorithm capable of accurately predicting the shelf-life of aquatic products in a variable-temperature environment in order to solve the above problem.

In Example 1, using sterilised shrimp paste as an experimental subject, the steps for shelf-life prediction were as follows.

Selecting shrimp paste (TVB-N of 43 mg/100 g, TPC of 50 CFU) to be examined.

Determination of TVB-N in sterilised shrimp paste was carried out as follows:
firstly, taking 10 g of sterilised shrimp paste, placing it in a distillation tube, adding 75 mL of distilled water and fully oscillate, then macerate for 30 min;
next, using the automatic Kjeldahl method, adding 1 g of magnesium chloride to the distillation tube, immediately connecting it to the distiller, and setting the parameters of the instrument as follows: boric acid acceptor 30 mL, distillation time 180 s or distillation volume 200 mL, hydrochloric acid of 0.1000 mol/L as the standard titration solution, and judging the end point by self-calibrating potentiometric titration (end point pH=4.65), and determining the end point after the measurement, and the instrument was cleaned in time;
the TVB-N in shrimp paste was calculated according to the following formula:

$$TVB - N ( mg / 100 g ) = \frac{( {V}_{1} - {V}_{2} ) \times c \times 14}{m} \times 100$$

where V₁ is the volume of hydrochloric acid standard titration solution consumed by the test solution, V₂ is the volume of hydrochloric acid standard titration solution consumed by the reagent blank, c is the concentration of hydrochloric acid standard titration solution, 14 is the mass of nitrogen equivalent to the titration of 1 mL of hydrochloric acid [c (HCl)=1.000 mol/L] standard titration solution, M is the mass of the sample, and 100 is the conversion factor.

Determination the TPC in sterilised shrimp paste TPC as follows:
firstly, taking 25 g of shrimp paste into a sterile homogenising bag containing 225 mL of saline, homogenising for 1˜2 min to make a 1:10 sample homogenate and preparing a diluted sample homogenate according to the 10-fold serial dilution method;
next, selecting 2˜3 dilutions of sample homogenate, aspirating 1 mL of sample homogenate in a sterile petri dish, pouring 46° C. counting agar medium onto the plate, and setting up a blank control, waiting for the agar to solidify and then placing it at 30° C. for 72 h;
then, recording the number of plate dilutions and the corresponding number of colonies, and selecting the plates with the TPC between 30 CFU and 300 CFU and with continuous dilution for counting, and calculating the TPC in the shrimp paste according to the following formula:

$$TPC ( C F U ) = \frac{Σ C}{( {n}_{1} + 0.1 {n}_{2} ) d}$$

where is the sum of the number of plate colonies, n1 is the number of low dilution plates, n2 is the number of high dilution plates, and d is the low dilution dilution factor;
placing the sterilised shrimp paste in a thermostat at 4° C., 8° C., 12° C., 16° C., 20° C., 22° C., 24° C., 28° C., 32° C., 36° C. and 40° C. respectively, and taking samples at 7:00 p.m. every day and determining the values of TPC and TVB-N of the shrimp paste samples at different temperatures in the aseptic environment, and stirring the samples thoroughly with sterile glass rods before sampling and ensuring that the sampling position and sampling quality are the same each time, and measuring the samples continuously for 60 days, at which time the training dataset contains a total of 1,320 time series data; the time series data is divided into training data set, validation data set and test data set in the ratio of 7:2:1 and standardized to establish the time series data set.

Define the Facformer model in Python™ 3.7 language environment, as shown in FIG. 1, the Facformer model mainly consists of three parts: data embedding layer, encoder and decoder.

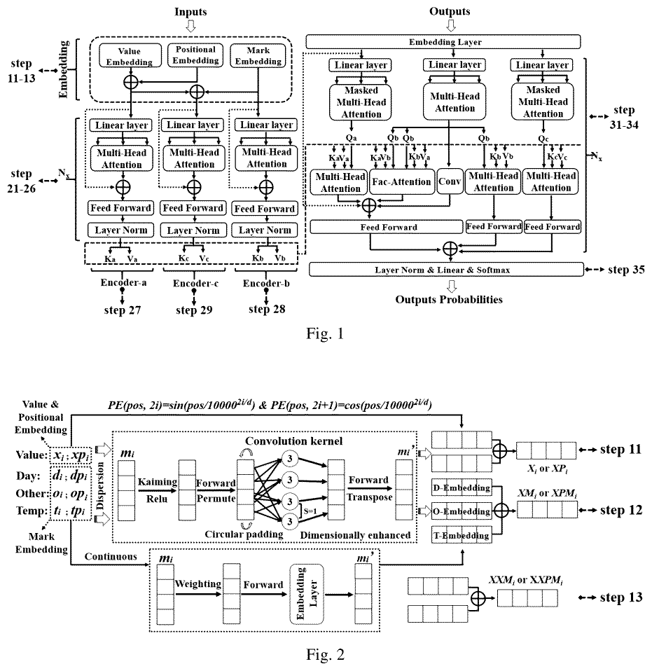

As shown in FIG. 2, the data embedding steps for the Facformer model are as follows:
the numerical embedding module is used to convert the measured values of TPC and TVB-N of aquatic products in the 1320 time-series data set into a matrix language that can be recognised by the model, and the positional embedding module is used to arrange the measured values of TPC and TVB-N of aquatic products according to the sequential order of the storage time in the matrix, which is converted into a matrix language with the characteristics of time series (step 10). If the measured data are discrete variables, the weight information of the measured values is transformed into the matrix language that can be recognised by the model through Relu loss function, feed-forward neural network and cyclic coding layer (step 11); if the measured data are continuous variables, the weight information of the measured values is directly transformed into the matrix language that can be recognised by the model through the feed-forward neural network and the embedding layer with linear transformation (step 12).

In summary, through the data embedding layer processing, the measured values with location information are transformed into Xᵢ feature matrix by the model (step 11), the values of days, temperature and other indicators are transformed into XMᵢ feature matrix by the model (step 12), and the values containing the measured values, days, temperature and other indicators are transformed into XXMᵢ feature matrix by the model (step 13), and finally, after the output of the encoder, the measured values, days, temperature and other indicator values are transformed into XPᵢ, XPMᵢ and XXPMᵢ feature matrices by the model.

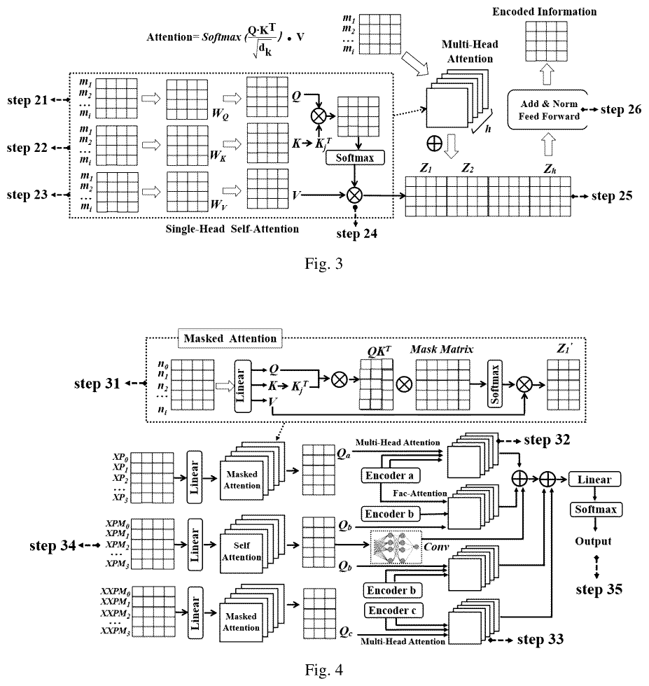

As shown in FIG. 3, the Encoder for the Facformer model works as follows:
firstly, the encoder of the Facformer model consists of three parallel encoder modules a, b and c, which are the inputs of the Facformer model; after the original time-series dataset is obtained from the feature matrices through the data embedding layer, the respective feature matrices are linearised into matrices Q (step 21), matrix K (step 22) and matrix V (step 23) through different linear transformation matrices W_{Q}, W_{K} and W_{V}, respectively, which are used to record their own feature information, and subsequently the model will perform dot product operation on matrix Q and matrix K to obtain the weight matrix containing the weights of the total number of aquatic product colonies TPC and TVB-N for scoring, and the weighting coefficients matrix is obtained by normalising and downscaling the weights matrix, multiplied by the matrix V corresponding to each position, and summed to obtain the output matrix encoded by the single-head self-attention mechanism (step 24);
finally, in order to increase the computing speed of the Facformer model, the output matrix Zᵢ obtained from a total of h parallel single-head self-attention mechanisms was summed up, i.e., a comprehensive encoded matrix was obtained after h parallel operations of the self-attention mechanisms, and the comprehensive encoded matrix was firstly uplifted by the fully-connected layer in the feed-forward neural network and downlifted by invoking the Relu function, and the downlifted matrix was then standardized based on the normalisation layer (step 25); After that, the normalised matrix is normalised based on the normalisation layer, and the robustness of the model is increased by adding the residual neural network and the original feature input matrix, and finally a matrix-encoded query dictionary of aquatic products time-series dataset is obtained, which contains the detailed weight information of aquatic products' storage time, storage temperature, TPC and TVB-N during the storage period (step 26);
where Xᵢ feature matrix is transformed by encoder-a to get Kₐ and Vₐ matrices (step 27); XMᵢ feature matrix is transformed by encoder-b to get K_{b} and V_{b} matrices (step 28). the XXMi feature matrix is encoder-c converted to obtain the K_{c} and V_{c} matrices for subsequent decoding in the decoder (step 29).

As shown in FIG. 4, the Decoder of the Facformer model works as follows:
firstly, Facformer's decoder consists of three parallel decoder modules, each containing at least two self-attention mechanisms for parsing the weight information of the matrix, and is the predictor and output side of the Facformer model;
next, the output matrices XPᵢ and XXPMᵢ obtained by the encoder are transformed into matrices Q, K and V through two different linear transformation layers, in which the matrix Q and K with the weight information of the samples will be masked by the masking matrix to remove the ambient noise that may affect the prediction results of the samples, and after dimensionality reduction, the masked prediction matrix is obtained (step 31);
then, the Qₐ matrix and Q_{c} matrix generated when the masking processed prediction matrix passes through the masking self-attention mechanism layer are passed downward, and the actual weight information of the data to be predicted is calculated by the second layer of the self-attention mechanism with the matrix K and matrix V passed in the encoder, and the actual weight information is compared with the query lexicon obtained from the encoding layer to obtain the prediction matrix; the Qₐ matrix generated by XPᵢ will be combined with the Kₐ matrix and Vₐ matrix passed by encoder-a for decoding of the message (step 32), and the Q_{c} matrix generated by XXPMᵢ will be combined with the K_{c} matrix and V_{c} matrix passed by encoder-c for decoding of the message (step 33);
after that, the output matrix XPMi obtained from the embedding layer for the number of days, temperature and other metrics is not masked with a masking matrix, and after it passes through the linear transformation layer, it passes directly through the self-attention mechanism layer to pass the Q_{b} matrix, which carries only the information about the future temperature without the rest of the ambient noise information, to the lower layer, and the Q_{b} matrix will be predicted by the three parallel arithmetic mechanisms to the quality metrics of the aquatic products in the future under the real variable-temperature conditions: firstly, the Q_{b} matrix will be combined using the matrix Kₐ passed in encoder-a and the matrix V_{b} passed in encoder-b through the Fac-self-attention mechanism, and at the same time, the matrix K_{b} passed in encoder-b and the matrix Vₐ passed in encoder-a will be used, and the combination of the two will be used to jointly compute the actual weight information of the data to be predicted, and the weight information will be used to compare with the query obtained from the encoding layer dictionary to obtain the prediction matrix; secondly, XPMᵢ uses a masking matrix to remove all the ambient noise through masking when inputting the first layer of multi-head self-attention mechanism, and the Q_{b} matrix generated when passing through the masking self-attention mechanism layer will be used to calculate the actual weight information of the data to be predicted through the second layer of the self-attention mechanism together with the matrix K_{b} and matrix V_{b} passed through the encoder, which will be used as a corrective matrix to increase the accuracy of the prediction; finally, the Q_{b} matrix is used for direct data fitting to the data to be predicted through convolutional neural networks and is used as a correction matrix to improve the accuracy of the model predictions (step 34);
finally, the output matrices XPi, XPMi and XXPMi obtained by the decoder will respectively elevate the dimensions of the matrices through the feed-forward neural network so as to make a comprehensive prediction of the data in a more detailed way, and the prediction weight information contained in the prediction matrices will be re-mapped to the time-series values through the normalisation layer, the linear transformation layer and the dimensionality reduction layer to obtain the prediction results, so as to complete the prediction of aquatic product TPC, TVB-N and shelf-life of aquatic products (step35).

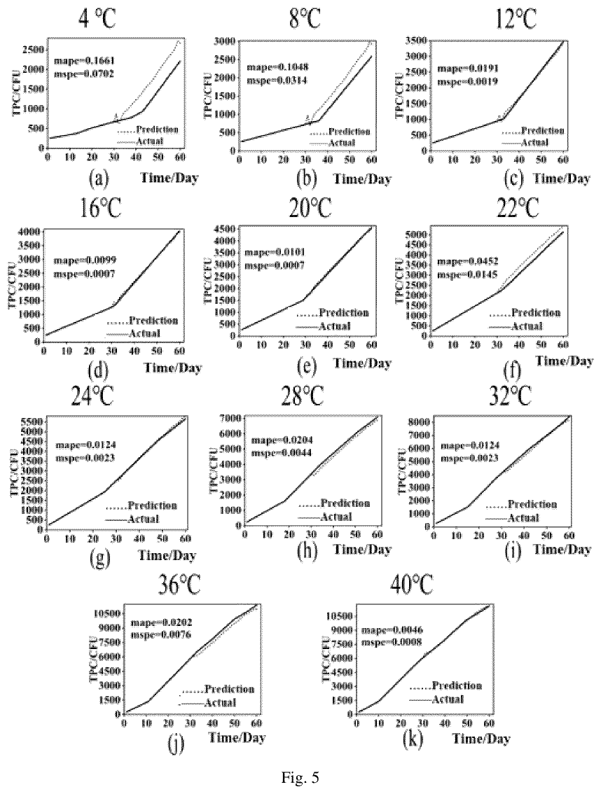

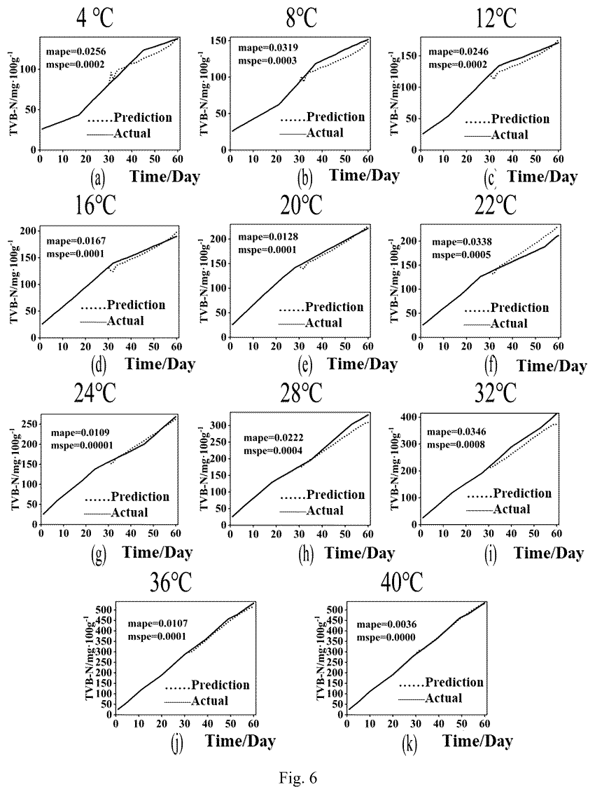

The initial model for deep learning of Facformer is constructed based on the nn.Module class contained in the Python™ library in the Python™ environment. The steps to determine the Facformer model are as follows:
inputing the training dataset into the Facformer model during the training phase to train the model hyperparameters;
inputing the validation dataset into the trained Facformer model in the validation phase to fine-tune the hyperparameters;
after fine-tuning, the parameters of the Facformor model are: the number of encoder layers is 2, the number of decoder layers is 1, the batch_size is set to 6, the optimiser learning rate is 0.0001, the trainepochs are 10, and the parameter earlystoppingpatience is set to 3. Also, the Gelu is set as the Facformer model's last activation function for the Forward layer;
the test dataset was fed into the constructed Facformer model during the testing phase and the model prediction performance was assessed using Mean Absolute Percentage Error (MAPE) and Mean Square Percentage Error (MSPE) and the results of the model prediction are shown in FIG. 5 and FIG. 6;

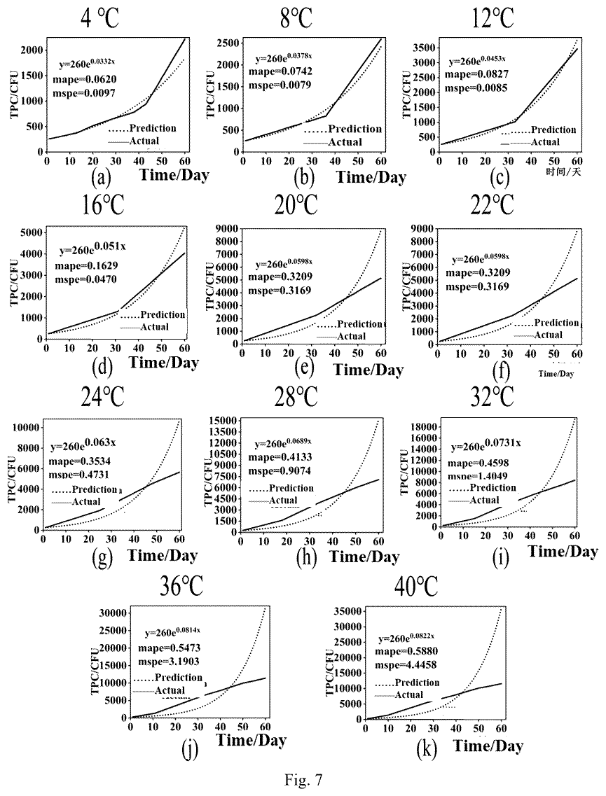

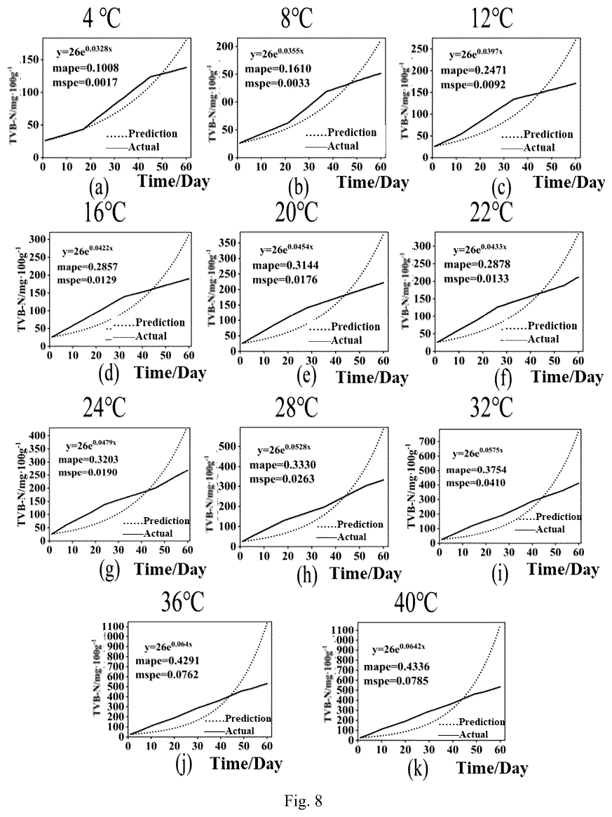

and comparing it with the prediction results of the traditional model, because the fitting coefficient of the zero-level equation constructed based on the TPC R²=10.6554, while the fitting coefficient of its first-level kinetic equation R² (10.6920) is larger than that of the zero-level equation, so here we chose to use the first-level kinetic equation to construct the prediction model based on the TPC, as shown in FIG. 7. Similarly, the first-order kinetic equations were chosen to construct a prediction model based on TVB-N, as shown in FIG. 8;

from FIGS. 5, 6, 7 and 8, it can be seen that although the prediction results of the conventional model are closer to the real values in the low-temperature environment, its gap in the high-temperature environment is particularly obvious and the prediction performance is poor. The Facformer model, on the other hand, significantly outperforms the traditional model in both low and high temperature environments, showing a strong prediction capability.

The prediction of quality indicators of sterilised shrimp paste in a variable temperature environment mainly comprises the following steps:
selecting a sample of shrimp paste to be predicted for sterilisation;
temperature, TPC and TVB-N of sterilised shrimp paste samples were determined based on steps 2 and 3 for 30 consecutive days and recorded as shown in Table 1;

TABLE 1

| | | TPC/CFU | | TVB-N/mg/100 g | |
|---|---|---|---|---|---|
| | | Real | Projected | Real | Projected |
| Temp/° C. | Time/day | value | value | value | value |
| 24 | 1 | 260.0000 | 260.0000 | 26.0000 | 26.0000 |
| 24 | 2 | 328.3333 | 328.3333 | 31.500 | 31.5000 |
| 24 | 3 | 398.3333 | 398.3333 | 37.0333 | 37.0333 |
| 24 | 4 | 483.3333 | 463.3333 | 42.6333 | 42.6333 |
| 24 | 5 | 553.3333 | 533.3333 | 47.9000 | 47.9000 |
| 24 | 6 | 605.0000 | 605.0000 | 58.4167 | 53.4167 |
| 24 | 7 | 673.3333 | 673.3333 | 58.9833 | 58.9833 |
| 24 | 8 | 785.3489 | 745.0000 | 70.4333 | 63.4333 |
| 24 | 9 | 813.3333 | 813.3333 | 68.0333 | 68.0333 |
| 24 | 10 | 880.0000 | 880.0000 | 72.4833 | 72.4833 |
| 24 | 11 | 991.6667 | 951.6667 | 79.9333 | 76.9333 |
| 24 | 12 | 1018.3333 | 1018.3333 | 81.4333 | 81.4333 |
| 24 | 13 | 1191.6667 | 1091.6667 | 85.9167 | 85.9167 |
| 24 | 14 | 1161.6667 | 1161.6667 | 98.4333 | 90.4333 |
| 24 | 15 | 1228.3333 | 1228.3333 | 98.7333 | 94.7333 |
| 24 | 16 | 1301.6667 | 1301.6667 | 99.2000 | 99.2000 |
| 24 | 17 | 1373.3333 | 1373.3333 | 108.4833 | 103.4833 |
| 24 | 18 | 1443.3333 | 1443.3333 | 110.9833 | 107.9833 |
| 24 | 19 | 1573.3333 | 1513.3333 | 113.1167 | 113.1167 |
| 24 | 20 | 1611.6667 | 1581.6667 | 117.9333 | 117.9333 |
| 24 | 21 | 1651.6667 | 1651.6666 | 123.3833 | 123.3833 |
| 24 | 22 | 1771.6667 | 1721.6667 | 130.2167 | 128.2167 |
| 24 | 23 | 1790.6667 | 1790.0000 | 153.2833 | 133.2833 |
| 24 | 24 | 1860.0000 | 1860.0000 | 138.3000 | 138.3000 |
| 24 | 25 | 1900.0000 | 1935.0001 | 161.1167 | 141.1167 |
| 24 | 26 | 2143.3333 | 2043.3333 | 164.2000 | 144.2000 |
| 24 | 27 | 2180.0000 | 2160.0000 | 157.0000 | 147.0000 |
| 24 | 28 | 2275.0000 | 2275.0000 | 150.1333 | 150.1333 |
| 24 | 29 | 2383.3333 | 2383.3333 | 153.0833 | 153.0833 |
| 24 | 30 | 2600.0000 | 2500.0000 | 175.5167 | 155.5167 |

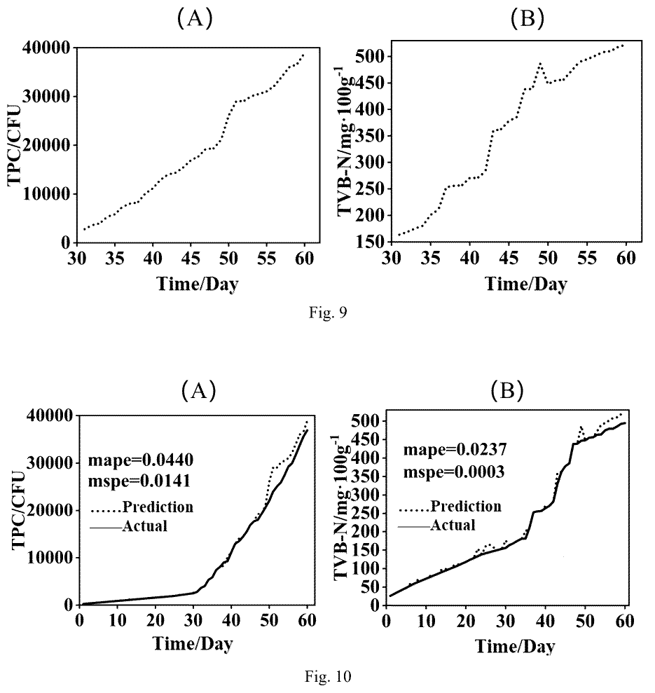

recording the temperature data of the sterilised shrimp paste from 30 to 60 days, import the temperature, the data of TPC and TVB-N during the consecutive 30 days, and the temperature data from 30 to 60 days into the pre-trained Facformer model for inference, and obtain the predicted values of TPC and TVB-N of the shrimp paste under the variable temperature condition. As shown in FIG. 9.

Selecting the corresponding national standard, local standard or enterprise standard according to the actual situation, and inputting into the Facformer model the limiting standards of volatile salt base nitrogen TVB-N and TPC of shrimp paste as specified in the domestic trade industry standard of the People's Republic of China, “Shrimp Sauce SB/T10525-2009” (450 mg/100 g and 8000 CFU). Comparison with Facformer's predicted TPC and TVB-N for shrimp paste revealed that the shelf life of sterilised shrimp paste based on TVB-N was 47 days and that of sterilised shrimp paste based on TPC was 38 days in a variable temperature environment.

To verify the predictive ability of the model in a variable temperature environment, the actual levels of TPC and TVB-N in the sterilised shrimp paste samples continued to be measured from day 30 to day 60 and compared to the levels predicted by Facformer. As shown in FIG. 10, it was found that the predictive ability of the Facformer model for TPC and volatile salt nitrogen TVB-N was not significantly different from the real values.

In Example 2, raw grass carp was used as an experiment to predict its shelf life.

Raw grass carp (TVB-N of 7 mg/100 g, TPC of 300 CFU) was selected for testing, skin, head, viscera and bone spurs were removed, and the edible part was taken and set aside for testing.

Determination of TVB-N in grass carp was carried out as follows:
firstly, taking 10 g of grass carp, placing it in a distillation tube, adding 75 mL of distilled water with sufficient oscillation and macerate for 30 min;
next, using the automatic Kjeldahl method, add 1 g of magnesium chloride to the distillation tube, immediately connecting it to the distiller, and setting the parameters of the instrument as follows: boric acid acceptor 30 mL, distillation time 180 s or distillation volume 200 mL, hydrochloric acid of 0.1000 mol/L as the standard titration solution, and judging the end point by self-calibrating potentiometric titration (end point pH=4.65), and determining the end point after the measurement, and the instrument was cleaned in time;
the TVB-N in grass carp was calculated according to the following formula:

$$TVB - N ( mg / 100 g ) = \frac{( {V}_{1} - {V}_{2} ) \times c \times 14}{m} \times 100 ,$$

where V₁ is the volume of hydrochloric acid standard titration solution consumed by the test solution, V₂ is the volume of hydrochloric acid standard titration solution consumed by the reagent blank, c is the concentration of hydrochloric acid standard titration solution, 14 is the mass of nitrogen equivalent to the titration of 1 mL of hydrochloric acid [c (HCl)=1.000 mol/L] standard titration solution, M is the mass of the sample, and 100 is the conversion factor.

Determination of TPC in grass carp was carried out as follows:
firstly, taking 25 g of grass carp into a sterile homogenising bag containing 225 mL of saline, homogenising for 1˜2 min to make a 1:10 sample homogenate and preparing a diluted sample homogenate according to the 10-fold serial dilution method;
next, selecting 2˜3 dilutions of sample homogenate, aspirating 1 mL of sample homogenate in a sterile petri dish, pouring 46° C. counting agar medium onto the plate, and setting up a blank control, waiting for the agar to solidify and then placing it at 30° C. for 72 h;
then, recording the number of plate dilutions and the corresponding number of colonies, and selecting the plates with TPC between 30 CFU and 300 CFU and with continuous dilution for counting, and the TPC in grass carp is calculated according to the following formula:

$$TPC ( C F U ) = \frac{Σ C}{( {n}_{1} + 0.1 {n}_{2} ) d} ,$$

where is the sum of the number of plate colonies, n₁ is the number of low dilution plates, n2 is the number of high dilution plates, and d is the low dilution dilution factor.

After the sterilised grass carp were cooled down to room temperature, they were placed in a thermostat at 4° C., 20° C., 36° C. and 40° C. respectively, and the samples were sampled and the values of TPC and TVB-N of the grass carp samples were determined in the aseptic environment at 7:00 p.m. in the evening of each day, Before sampling, a sterile glass rod was used to stir the samples thoroughly and ensure that the sampling location and sampling quality were consistent each time, and the samples were measured continuously for 30 days, at which time the training dataset contained a total of 240 time-series data; the time series data is divided into training data set, validation data set and test data set in the ratio of 7:2:1 and standardized to establish the time series data set.

Define the Facformer model in Python™ 3.7 language environment, as shown in FIG. 1, the Facformer model mainly consists of three parts: data embedding layer, encoder and decoder.

As shown in FIG. 2, the data embedding steps for the Facformer model are as follows:
the numerical embedding module is used to convert the measured values of TPC and TVB-N of grass carp in the 240 time-series data set into a matrix language that can be recognised by the model, and the positional embedding module is used to arrange the measured values of TPC and TVB-N of aquatic products according to the sequential order of the storage time in the matrix, which is converted into a matrix language with the characteristics of time series (step 10). If the measured data are discrete variables, the weight information of the measured values is transformed into the matrix language that can be recognised by the model through Relu loss function, feed-forward neural network and cyclic coding layer (step 11); if the measured data are continuous variables, the weight information of the measured values is directly transformed into the matrix language that can be recognised by the model through the feed-forward neural network and the embedding layer with linear transformation (step 12).
in summary, through the data embedding layer processing, the measured values with location information are transformed into Xᵢ feature matrix by the model (step 11), the values of days, temperature and other indicators are transformed into XMᵢ feature matrix by the model (step 12), and the values containing the measured values, days, temperature and other indicators are transformed into XXMᵢ feature matrix by the model (step 13), and finally, after the output of the encoder, the measured values, days, temperature and other indicator values are transformed into XPᵢ, XPMᵢ and XXPMᵢ feature matrices by the model.

As shown in FIG. 3, the encoder for the Facformer model works as follows:
firstly, the encoder of the Facformer model consists of three parallel encoder modules a, b and c, which are the inputs of the Facformer model; after the original time-series dataset is obtained from the feature matrices through the data embedding layer, the respective feature matrices are linearised into matrices Q (step 21), matrix K (step 22) and matrix V (step 23) through different linear transformation matrices W_{Q}, W_{K} and W_{V}, respectively, which are used to record their own feature information, and subsequently the model will perform dot product operation on matrix Q and matrix K to obtain the weight matrix containing the weights of the total number of aquatic product colonies TPC and TVB-N for scoring, and the weighting coefficients matrix is obtained by normalising and downscaling the weights matrix, multiplied by the matrix V corresponding to each position, and summed to obtain the output matrix encoded by the single-head self-attention mechanism (step 24);
finally, in order to increase the computing speed of the Facformer model, the output matrix Zᵢ obtained from a total of h parallel single-head self-attention mechanisms was summed up, i.e., a comprehensive encoded matrix was obtained after h parallel operations of the self-attention mechanisms, and the comprehensive encoded matrix was firstly uplifted by the fully-connected layer in the feed-forward neural network and downlifted by invoking the Relu function, and the downlifted matrix was then standardized based on the normalisation layer (step 25). After that, the normalised matrix is normalised based on the normalisation layer, and the robustness of the model is increased by adding the residual neural network and the original feature input matrix, and finally a matrix-encoded query dictionary of aquatic products time-series dataset is obtained, which contains the detailed weight information of aquatic products' storage time, storage temperature, TPC and TVB-N during the storage period (step 26).
where Xᵢ feature matrix is transformed by encoder-a to get Kₐ and Vₐ matrices (step 27); XMᵢ feature matrix is transformed by encoder-b to get K_{b} and V_{b} matrices (step 28). the XXMᵢ feature matrix is encoder-c converted to obtain the K_{c} and V_{c} matrices for subsequent decoding in the decoder (step 29).

As shown in FIG. 4, the decoder of the Facformer model works as follows:
firstly, Facformer's decoder consists of three parallel decoder modules, each containing at least two self-attention mechanisms for parsing the weight information of the matrix, and is the predictor and output side of the Facformer model;
next, the output matrices XPᵢ and XXPMᵢ obtained by the encoder are transformed into matrices Q, K and V through two different linear transformation layers, in which the matrix Q and K with the weight information of the samples will be masked by the masking matrix to remove the ambient noise that may affect the prediction results of the samples, and after dimensionality reduction, the masked prediction matrix is obtained (step 31);
then, the Qₐ matrix and Q_{c} matrix generated when the masking processed prediction matrix passes through the masking self-attention mechanism layer are passed downward, and the actual weight information of the data to be predicted is calculated by the second layer of the self-attention mechanism with the matrix K and matrix V passed in the encoder, and the actual weight information is compared with the query lexicon obtained from the encoding layer to obtain the prediction matrix; the Qₐ matrix generated by XPᵢ will be combined with the Kₐ matrix and Vₐ matrix passed by encoder-a for decoding of the message (step 32), and the Q_{c} matrix generated by XXPMᵢ will be combined with the K_{c} matrix and V_{c} matrix passed by encoder-c for decoding of the message (step 33);
after that, the output matrix XPMᵢ obtained from the embedding layer for the number of days, temperature and other metrics is not masked with a masking matrix, and after it passes through the linear transformation layer, it passes directly through the self-attention mechanism layer to pass the Q_{b} matrix, which carries only the information about the future temperature without the rest of the ambient noise information, to the lower layer, and the Q_{b} matrix will be predicted by the three parallel arithmetic mechanisms to the quality metrics of the aquatic products in the future under the real variable-temperature conditions: firstly, the Q_{b} matrix will be combined using the matrix Kₐ passed in encoder-a and the matrix V_{b} passed in encoder-b through the Fac-self-attention mechanism, and at the same time, the matrix K_{b} passed in encoder-b and the matrix Vₐ passed in encoder-a will be used, and the combination of the two will be used to jointly compute the actual weight information of the data to be predicted, and the weight information will be used to compare with the query obtained from the encoding layer dictionary to obtain the prediction matrix; secondly, XPMᵢ uses a masking matrix to remove all the ambient noise through masking when inputting the first layer of multi-head self-attention mechanism, and the Q_{b} matrix generated when passing through the masking self-attention mechanism layer will be used to calculate the actual weight information of the data to be predicted through the second layer of the self-attention mechanism together with the matrix K_{b} and matrix V_{b} passed through the encoder, which will be used as a corrective matrix to increase the accuracy of the prediction; finally, the Q_{b} matrix is used for direct data fitting to the data to be predicted through convolutional neural networks and is used as a correction matrix to improve the accuracy of the model predictions (step 34);
finally, the output matrices XPᵢ, XPMᵢ and XXPMᵢ obtained by the decoder will respectively elevate the dimensions of the matrices through the feed-forward neural network so as to make a comprehensive prediction of the data in a more detailed way, and the prediction weight information contained in the prediction matrices will be re-mapped to the time-series values through the normalisation layer, the linear transformation layer and the dimensionality reduction layer to obtain the prediction results, so as to complete the prediction of aquatic product TPC, TVB-N and shelf-life of aquatic products (step35).

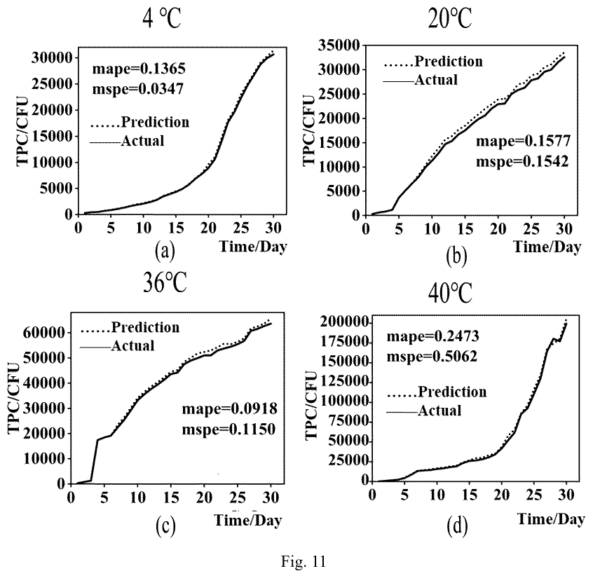

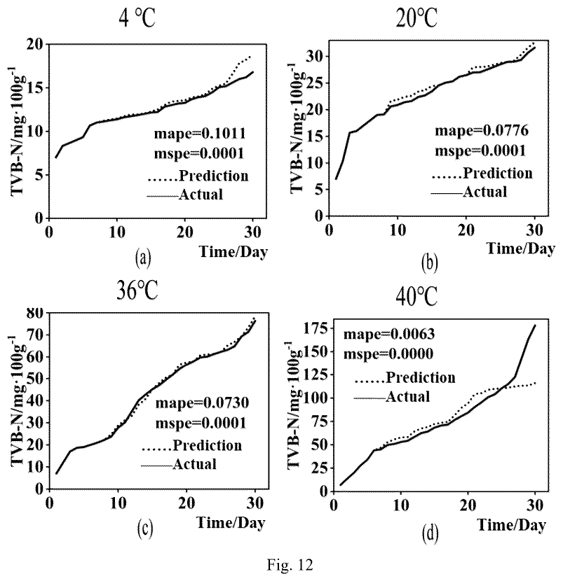

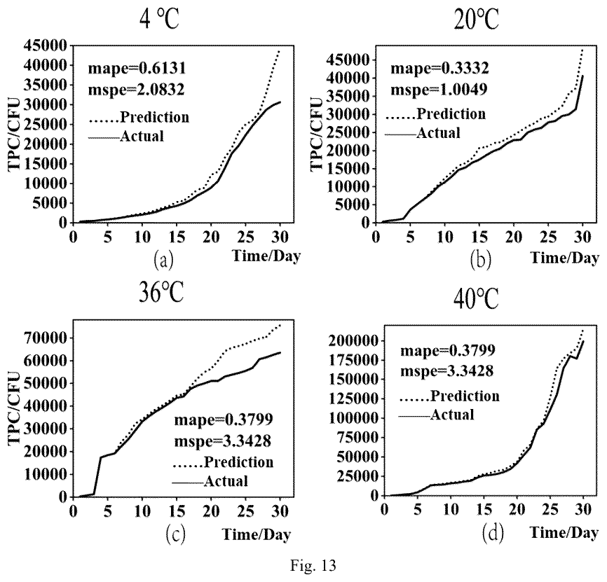

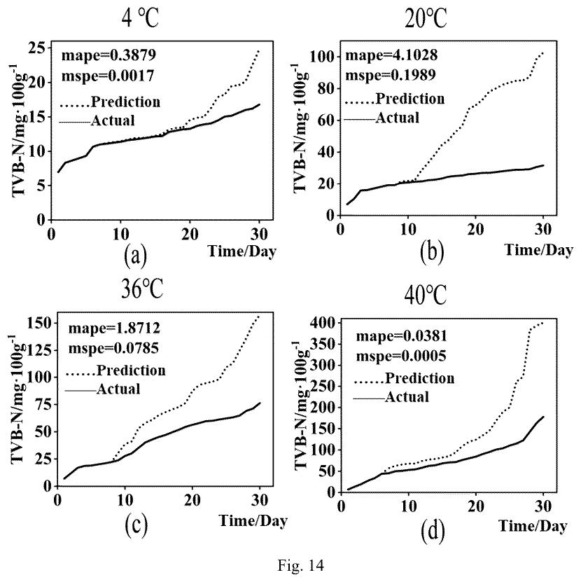

The initial model for deep learning of Facformer is constructed based on the nn.Module class contained in the Python™ library in the Python™ environment. The steps to determine the Facformer model are as follows:
the training dataset is fed into the Facformer model during the training phase to train the model hyperparameters;
the validation dataset is fed into the trained Facformer model during the validation phase to fine-tune the hyperparameters;
after fine-tuning, the parameters of the Facformor model are: the number of encoding layers is 2, the number of decoding layers is 1, batch_size is set to 6, the optimiser learning rate is 0.0001, and the trainepochs are 10, The parameter earlystoppingpatience is set to 3, while Gelu is set as the activation function for the final Forward layer of the Facformer model;
the test dataset was input into the constructed Facformer model during the testing phase and the model prediction performance was evaluated using Mean Absolute Percentage Error (MAPE) and Mean Square Percentage Error (MSPE) and the results of the model prediction are shown in FIG. 11 and FIG. 12;
and compared them with the predictions of the conventional model, since the fit coefficient of the zero-level equation constructed on the basis of the TPC, R²=10.7321. And the fitting coefficient R² (10.7439) based on the first-order kinetic equation is larger than the zero-order equation, so here we chose to use the first-order kinetic equation to construct the prediction model based on the TPC, as shown in FIG. 13. Similarly, the first-order kinetic equations were chosen to construct a prediction model based on TVB-N, as shown in FIG. 14;
from FIGS. 11, 12, 13 and 14, it can be seen that although the prediction results of the conventional model are closer to the true values in the low-temperature environment, its gap in the high-temperature environment is particularly obvious and the prediction performance is poor. The Facformer model, on the other hand, significantly outperforms the traditional model in both low and high temperature environments, showing a strong prediction capability.

The prediction of quality indicators of grass carp in a variable temperature environment mainly comprises the following steps:
the selection of samples of grass carp to be predicted;
temperature, TPC and TVB-N of grass carp samples were determined based on steps 2 and 3 over a period of 7 consecutive days and recorded as shown in Table 2;

TABLE 2

| Temp/ | Time/ | TPC/CFU | | TVB-N/mg/100 g | |
|---|---|---|---|---|---|
| ° C. | day | Real value | Projected value | Real value | Projected value |
| 20 | 1 | 300.0000 | 300.0000 | 7.0000 | 7.0000 |
| 20 | 2 | 605.5000 | 605.5000 | 10.3333 | 10.3333 |
| 20 | 3 | 804.3571 | 804.3571 | 15.6666 | 15.6666 |
| 20 | 4 | 1147.8412 | 1147.8412 | 16.0000 | 16.0000 |
| 20 | 5 | 3640.9857 | 3640.9857 | 17.0000 | 17.0000 |
| 20 | 6 | 5143.1643 | 5143.1643 | 18.0000 | 18.0000 |
| 20 | 7 | 6562.5638 | 6562.5638 | 19.0000 | 19.0000 |

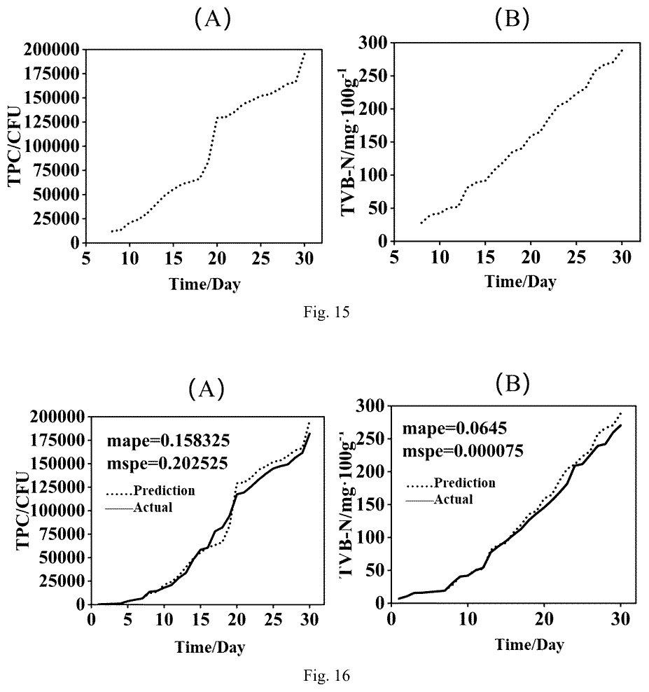

recording the temperature data of grass carp from 7 to 30 days, and import the temperature, data of TPC and TVB-N in 7 consecutive days, and temperature data from 7 to 30 days into the pre-trained Facformer model for inference, and obtain the predicted values of TPC and TVB-N of grass carp under variable temperature conditions, as FIG. 15 shown.

Select the corresponding national standard, local standard or enterprise standard according to the actual situation, and input the limiting standards of TVB-N and TPC of grass carp specified in the national standard for food safety of the People's Republic of China for animal aquatic products “GB10136-2015” (30 mg/100 g and 10,000 CFU) into the Facformer model. Comparison with Facformer's predicted TPC and TVB-N for grass carp revealed that the shelf life of grass carp in a variable temperature environment was 11 days based on TVB-N, while the shelf life of grass carp based on TPC was 7 days.

To verify the predictive ability of the model in a variable temperature environment, the actual levels of TPC and TVB-N in the sterilised shrimp paste samples continued to be measured from day 7 to day 30 and compared to the levels predicted by Facformer. As shown in FIG. 16, it was found that the predictive ability of the Facformer model for TPC and volatile salt nitrogen TVB-N was not significantly different from the real values.

Although the present application has been disclosed as above by way of example, it is not intended to limit the present application, any person skilled in the art, without departing from the spirit and scope of the present application, when some changes and embellishments can be made, so that the scope of protection of the present application shall be subject to the definition of the claims.

The invention claimed is:

1. A method for high-precision prediction of shelf-life of aquatic products in a variable-temperature environment, comprising:
pre-constructing and training a Facformer model for predicting shelf life of an aquatic product;
obtaining the aquatic product to be predicted;
measuring and recoding temperature, TPC (Total Plate Count) and TVB-N (Total Volatile Base-Nitrogen) of the aquatic product during a total of p consecutive days;
recording temperature data of the aquatic product from p to q days where q is greater than p, and importing the data of the temperature, TPC and TVB-N in a total of p consecutive days, and the temperature data from p to q days into pre-trained Facformer model for inference, obtaining predicted values of the TPC and TVB-N of the aquatic product under variable temperature conditions;
inputting limiting standards for TVB-N and TPC in the aquatic product into the pre-trained Facformer model, and comparing output from the Facformer model of the limiting standards for TVB-N and TPC in the aquatic product with predicted TPC and TVB-N of the aquatic product to obtain a predicted value of the shelf-life of the aquatic product under variable temperature conditions;
wherein pre-constructing and training the Facformer model comprises:
preparing the aquatic product by removing shell, viscera or bone spurs of the aquatic product to obtain an edible part;
placing samples of the prepared aquatic product in constant temperature incubators at m random temperatures in a range from 4° C. to 40° C., sampling and determining the TPC and TVB-N of the samples under variable temperature conditions at a fixed time every day for n consecutive days, to obtain a time series of data comprising m×n×2 entries;
dividing the time series data into a training dataset, a validation dataset, and a test dataset, and standardizing the training dataset, the validation dataset, and the test dataset to establish a time series dataset; and
defining the Facformer model, wherein the Facformer model mainly consists of three parts: a data Embedding Layer, an Encoder and a Decoder;
wherein data Embedding steps for the Facformer model are as follows:
randomly vectorizing measured values of the TPC and TVB-N in the time series data by Value embedding, recovering time series information of the aquatic product in storage process by using Position embedding, and upgrading and summing up the measured values of the TPC and TVB-N to obtain a Basic embedding while transforming the time series data into a feature matrix; and
adding temperature information of the aquatic product in the storage process by Mark embedding, transforming the temperature data into a feature matrix, and summing up a Basic embedding and a Mark embedding to obtain a Total embedding, which is then transformed into a feature matrix;
wherein the Encoder for the Facformer model is established as follows:
calculating weight coefficients of a feature matrix obtained by the Basic embedding, the Mark embedding and the Total embedding, respectively, by three parallel Encoder blocks of an input of the Facformer model;
linearizing corresponding input matrix into a Query matrix (Q), a Key matrix (K) and a Value matrix (V) via different weighting matrices of W_{Q}, W_{K} and W_{V}, respectively, and performing dot product operation on matrix Q and matrix K to obtain corresponding scores of TPC and TVB-N of the aquatic product;
normalizing the scores with a softmax activation function to obtain a matrix of weighting coefficients, which is multiplied by the matrix V corresponding to each position and summed up to get an output matrix of an Attention layer; and
obtaining a Attention matrix obtained by a multi-head self-attention mechanism, which is normalized by an Add & Normalize grid layer, summing with a feature matrix in front of the Attention layer via a residual neural network, and downgrading by invoking a Relu function after being upgraded by a fully-connected layer in a Feed Forward layer to obtain the Encoder containing weights corresponding to time-temperature-TPC-TVB-N in the input matrix;
wherein the Decoder for the Facformer model is established as follows:
masking time series data of a prediction matrix by a Mask matrix when inputting a first layer of Multi-Head Attention, the prediction matrix is obtained based on the Basic embedding and the Total embedding; and an output of the Facformer model comprising three parallel Decoder blocks, each Decoder block contains two layers of the Multi-Head Attention;
passing down the Q matrix generated when the Masked prediction matrix passes through a Masked-Multi-Head Attention layer, and computing the actual weights of a timing data to be predicted in a second Multi-Head Attention layer together with the matrix K and the matrix V passed down from the Encoder, and comparing the actual weights with the Encoder;
instead of masking the prediction matrix obtained by Mark embedding with the Mask matrix, passing the Q matrix with future temperature information directly onward to lower layers, and completing a decoding of this part of the time series data through a Fac Attention mechanism; and
a residual neural network and synergistic prediction of the Facformer model comprises: summing up on an upgraded dimension an original input matrix (XP) based on a residual neural network of aquatic product time-series dataset, a prediction matrix (XP1) based on Masked-Multi-Head Attention, a prediction matrix (XP2) based on a convolutional neural network of experimental results with direct data fitting, and a prediction matrix (XP3) obtained by the Fac Attention mechanism, while jointly decoding real weighting information about quality of the aquatic product under variable temperature conditions in the future by means of collaboratively predicted Mark data XPM″ and XXPM″, and finally remapping a weighting information into real time series data by means of a fully connected neural network and a softmax layer;
wherein the Facformer model is determined by following steps:
inputting a training dataset into the Facformer model during a training phase to train model hyperparameters;
fine-tuning hyperparameters by inputting the validation dataset into a trained Facformer model during a validation phase; and
inputting the test dataset into a constructed Facformer model during a testing phase, evaluating model prediction performance using MAE (Mean Absolute Error), MSE (Mean Square Error), RMSE (Root Mean Square Error), MAPE (Mean Absolute Percentage Error), and MSPE (Mean Square Percentage Error), and obtaining the Facformer model.

2. The method for high-precision prediction of shelf-life of aquatic products in a variable-temperature environment according to claim 1, wherein the process of determining TVB-N in aquatic products is carried out in the following steps:
taking 10 g of the aquatic product and placing the aquatic product in a distillation tube, adding 75 mL of distilled water to the distillation tube, and immersing the aquatic product for 30 min after sufficient oscillation;
measuring nitrogen content of the aquatic product by automatic Kjeldahl nitrogen determination method, wherein 1 g of magnesium chloride is added to the distillation tube, which is immediately connected to a distiller, and parameters of apparatus are set as follows: boric acid receiving solution 30 mL, distillation time 180 s or distillation volume 200 mL, 0.1000 mol/L hydrochloric acid as standard titration solution, determining an end point by self-calibrating potentiometric titration where the end point occurs when pH=4.65, and cleaning the apparatus after measurement;
calculating TVB-N in the aquatic product according to the following formula:
$$TVB - N ( mg / 100 g ) = \frac{( {V}_{1} - {V}_{2} ) \times c \times 14}{m} \times 100 ,$$
wherein V₁ is a volume of hydrochloric acid standard titration solution consumed by the test solution, V₂ is a volume of hydrochloric acid standard titration solution consumed by the reagent blank, c is a concentration of hydrochloric acid standard titration solution, 14 is a mass of nitrogen corresponding to the consumption of 1 mL of hydrochloric acid [c (HCl)=1.000 mol/L] standard titration solution, M is the mass of a sample of the samples, and 100 is a conversion factor.

3. The method for high-precision prediction of shelf-life of aquatic products in a variable-temperature environment according to claim 1, wherein the process of determining the TPC of the aquatic product is carried out in the following steps:
putting 25 g of the aquatic product into a sterile homogenization bag containing 225 mL of saline, and homogenizing for 1-2 min to make a 1:10 sample homogenate, which is diluted according to a 10-fold series dilution method to obtain a diluted sample homogenate;
selecting a sample homogenate obtained by diluting for 2-3 times, pipetting 1 mL of the sample homogenate into a sterile petri dish, into which count agar medium at 46° C. is poured to obtain a pour plate culture, and placing the petri dish at 30° C. to incubate for 72 h after the agar is solidified and setting up a blank control;
recording the dilution multiplicities of the plate and corresponding number of colonies, selecting plates with a total number of colonies between 30 CFU and 300 CFU and with continuous dilution multiplicities for counting, and calculating total number of colonies of the aquatic product according to the following formula:
$$TPC ( C F U ) = \frac{Σ C}{( {n}_{1} + 0.1 {n}_{2} ) d}$$
wherein ΣC is a sum of a number of plate colonies, n1 is a number of plates at a first dilution scale (low dilution multiplicity), n2 is a number of plates at a second dilution scale (high dilution multiplicity), and d is a first dilution scale dilution factor.
